(19) United States
(12) Patent Application Publication
(10) Pub. No.: US 2018/0264132 A1
(43) Pub. Date: Sep. 20, 2018

(54) Combined Tissue Resonance Suppression Therapy and Autologous Polyvalent Biological Vaccine

(71) Applicants: David Michaeli, Ashkelon (IL); Orna Berry, Tel Aviv (IL)

(72) Inventor: David Michaeli, Ashkelon (IL)

(21) Appl. No.: 15/462,857

(22) Filed: Mar. 19, 2017

(51) Int. Cl. A61K 47/68; C07K 14/47; C07K 16/30; C07K 16/42; G01N 33/574

(52) U.S. Cl. CPC A61K 47/6873; C07K 14/47; A61K 38/00; C07K 16/42; G01N 33/574; C07K 16/30

(57) ABSTRACT

A method for strengthening the immune response of a weakened immune system, wherein the method includes providing a doses of tissue resonance suppression therapy to a patient and injecting an autologous polyvalent biological vaccine to the patient, wherein the autologous polyvalent biological vaccine was produced from the patient venous blood, a system for performing tissue resonance suppression therapy, a system and a process for the production of the autologous polyvalent biological vaccine for treating cancer, and an autologous polyvalent biological vaccine for personalized and precision medicinal, for treatment of individual cancerous patients, wherein producing a new dose of autologous polyvalent biological vaccine is done from a patient venous blood that was taken after time interval from the time in which a previous venous blood was taken, wherein the vaccine is adapted for treating the individual cancerous patients at a specific stage of his disease, in accordance with embodiments of the present invention.

### FIELD OF THE INVENTION

The present invention relates to the treatment of cancer, more particularly, the present invention relates to combined Tissue Resonance Suppression Therapy (TRST) and Autologous Polyvalent Biological Vaccine (APBV).

### BACKGROUND OF THE INVENTION

According to “What is the second biggest cause of lung cancer in the world?” (https://www.quora.com/What-is-the-second-biggest-cause-of-lung-cancer-in-the-world), “cancer” is a generic term for a group of diseases that can manifest in any part of the body, also referred to as “malignant tumors” or “neoplasms”. This group of diseases has the common defining feature of rapid growth of abnormal cells beyond normal boundaries, which can proceed to invade adjacent organs and spread throughout the body, a process referred to as “metastasizing”, which is a major cause of death from cancer.

Cancer caused 8.2 million deaths worldwide in 2012 alone and is a leading cause of death, with the most common fatal cancers being lung, liver, stomach, colorectal, breast, and esophageal cancer.

Cancer starts with a single cell, which transforms into a tumor cell through a multistage process of progression from a precancerous lesion to malignant tumors. This process is the result of interaction between individual genetic makeup and three types of external factors, including: physical carcinogens, e.g. ionizing and ultraviolet radiation; chemical carcinogens, e.g. asbestos, chemicals in tobacco smoke, aflatoxin, arsenic, etc.; and biological carcinogens, such as viral or bacterial infections or parasites.

Another crucial factor in the development of cancer is aging. The risk factors for numerous specific types of cancer increase with age, therefore the incidence of cancer increases significantly with age. This increase in risk is compounded with less effective cellular repair mechanisms in the aging body.

The main risk factors for cancer tobacco consumption, alcohol consumption, imbalanced diet, and physical inactivity, as well as chronic infections particularly common in less developed countries. Hepatitis B and C increase the risk for liver cancer, while particular strains of Human Papilloma Virus (HPV) increase the risk for cervical cancer. Likewise, Human Immunodeficiency Virus infection increase the risk for numerous types of cancer, including cervical cancer.

Reduction of the damage from cancer can be achieve through application of the extensive knowledge and methods of intervention for disease management and prevention presently available. Many types of cancer nowadays have a very high recovery rate thanks to implementation of evidence-based strategies for prevention, early detection, and disease management.

Over 30% of cancer deaths could be averted through modification of key risk factors, such as tobacco use, obesity, imbalanced diet, lack of physical activity, alcohol consumption, sexually transmitted infections, ionizing and non-ionizing radiation, environmental pollution, etc. Tobacco is presently the single most significant risk factor for cancer, resulting in approximately 20% of worldwide cancer fatalities, and 70% of worldwide lung cancer fatalities. In many underdeveloped countries, up to 20%/o of cancer fatalities are caused by Hepatitis B and Human Papilloma virus infections.

Known prevention strategies include: avoiding or minimizing the aforementioned risk factors; vaccination against HPV and Hepatitis B virus; minimizing occupational hazards; minimizing exposure to non-ionizing radiation of sunlight; and minimizing exposure to ionizing radiation (occupational or from medical diagnostic imaging).

According to an additional online publication titled: “Global Urological Cancer Market to 2022—Strong Growth Driven by Rising Prevalence, Increased Uptake of Hormone Therapies and Approval of Novel Biologics”, (http://www.prnewswire.com/news-releases/global-urological-cancer-market-to-2022---strong-growth-driven-by-rising-prevalence-increased-uptake-of-hormone-therapies-and-approval-of-novel-biologics-300343097.html):

“A number of common etiologic factors have been strongly characterized as raising the risk of developing urological cancers, including age, chronic inflammation, gender, obesity, tobacco usage and heritable cancer syndromes. The risk of cancer increases greatly in patients over the age of 65. Populations in developed countries are projected to become increasingly aged and show rising obesity incidence, which will drive both cancer prevalence and revenue growth for its treatments.”

While according to “Global Cancer Vaccines Market to Reach $7.5 Billion by 2022 at a CAGR of 16.93%” (http://www.prnewswire.co.uk/news-releases/global-cancer-vaccines-market-to-reach-75-billion-by-2022-at-a-cagr-of-1693-603540446.html):

“Cancer vaccines are being developed as a method of preventing certain types of cancer, and as therapeutic treatments to treat existing cancers across a range of indications in oncology, either as stand-alone therapies or in combination with traditional cancer therapeutics such as chemotherapy and surgery. The high mortality rate associated with cancer and its resistance to conventional treatments such as radiation and chemotherapy has led to the investigation of a variety of anti-cancer immunotherapies, which have a lower toxicity associated with their use than traditional chemotherapies. Therapeutic vaccine administration will increase the overall survival of poor-performance-status patients, and enable more rounds of treatment to be given—factors that will contribute to growing global revenues for this class of therapy. However, cancer vaccines are not perceived as having strong commercial potential, as immune checkpoint inhibitors are expected to dominate the treatment landscape for leukemia and lymphoma during the forecast period.”

Furthermore, in “Pharmacogenetics-Guided Dosing for Fluoropyrimidines in Cancer Chemotherapy” by Zhi-Xu of Guiyang Medical University and Shu-Feng Zhou of the University of South Florida, cites cancer as a leading cause of global mortality.

“Worldwide, almost 32.5 million people diagnosed with cancer within the five years previously were alive at the end of 2012. By 2030, the global burden is expected to grow to 21.4 million new cancer cases and 13.2 million cancer deaths simply due to the growth and aging of the population, as well as reduction in childhood mortality and deaths from infectious diseases in developing countries.”

The field of immuno-oncology generally holds that cancer cells often have molecules known as “cancer-specific antigens” on their surface, as opposed to healthy cells. In the bloodstream, these molecules act as antigens, stimulating the immune system to create antibodies to recognize and destroy the corresponding cancer cells. All cancer vaccines use this approach, however developing effective vaccines is difficult because:

a. Cancer causes suppression of the immune system. Present research uses adjuvants to overcome this suppression.

b. Cancer cells are mutations of the patient's own healthy cells. Therefore, the immune system may not necessarily recognize them as harmful and ignore them rather than destroying them.

c. Tumors larger than 5 cm in diameter or in advanced stages (stages III or IV) will not necessarily be fully destroyed by use of a vaccine.

d. Patients with background illness or of old age can have weaker immune systems, and therefore their immune systems can respond less effectively to vaccination.

e. Many conventional cancer treatments cause further suppression of the immune system.

In spite of all the known and existent solutions, there is a need for alternative therapies to overcome or mitigate at least some of the deficiencies of the prior art.

Because of these reasons, some researchers think cancer treatment vaccines may work better for smaller tumors, such as tumors less than 5 centimeters in diameter, or early-stage cancers namely stages I and II.

A. Nencioni, F. Grinebach, F. Patrone & P. Brossart, in their paper, “Anticancer Vaccination Strategies”. (Annals of Oncology 15 (Supplement 4): iv153-iv160, 2004 doi:10.1093/annonc/mdh920), http://annonc.oxfordjournals.org/content/15/suppl_4/iv153.full.pdf, a survey report which also teach:

a. That the injection of whole tumor cells or tumor lysates of allogenic source for the induction of antitumor immune response results in stabilization of disease in only 10-20% of the patients that received the injection.

b. That the median survival of patients after allogenic tumor vaccine treatment was 56.4 months, versus 31.9 months in the non-vaccine group. This kind of allogenic vaccines has disadvantages which include: low effectivity and unpleasant immune reactions of the body to the vaccine treatment, host (vaccine) and recipient (patient) relations.

c. That antitumor vaccination studies so far tend to use autologous tumor material. Tumor cells can be engineered to express immune-stimulatory factors, such as Inter Leukin-2 (IL-2), Inter Leukin-4 (IL-4), Granulocyte Macrophage—Colony Stimulating factor (GM-CSF). Wherein the Inter Leukin-2 is a type of cytokine signaling molecule in the immune system, a protein that regulates the activities of white blood cells that are responsible for immunity, wherein the Inter Leukin-4 is a cytokine that induces differentiation of naive helper Tₕ cells to Tₕ2 cells (T helper cells 2), wherein the T cell is a type of lymphocyte that plays a central role in cell-mediated immunity, and wherein the T helper cells are a type of T cell that play an important role in the immune system. Wherein Granulocyte Macrophage—Colony-Stimulating Factor (GM-CSF) is a monomeric glycoprotein secreted by macrophages, T cells, mast cells, NK cells (Natural Killer cells) endothelial cells and fibroblasts that functions as a cytokine, wherein the NK cells have the ability to recognize stressed cells in the absence of antibodies and Major Histocompatibility Complex (MHC), allowing for a much faster immune reaction.

Immunotherapy given to patients at stage II of disease was associated with a significantly longer recurrence free period (with a probability index of P=0.011) and 61% of tumor risk reduction for recurrence, but immunotherapy given to patients at stage III, showed no significant benefit of vaccination.

As used herein the specification and in the claims section that follows, the terms “stage I, stage II, stage III, and stage IV” and the like refer to the classification of stages of tumor development used in modern oncology.

All cancer patients have a suppressed immune system due to chemotherapy, radiation therapy, and due to the immune system's natural battle against cancerous cells.

The aforementioned effects sometimes result in Anti-Cancer Vaccines Therapy (ACVT) working slowly and not effective.

In the conventional triad of treatment of cancer patients: a. surgical excision of tumor; b. chemo; and c. X-ray Therapy, the surgical procedure often results in Surgical Stress Induced (SSI) Tumor Cell Mutations (TCM). Post operational residual tumor cells on the bed of the tumor site, rapidly go through mutations and recurrent growth. Due to this effect, resistance is developed by the cells to the chemotherapy drugs and vaccine therapy, causing spreading of mutated tumor cell free DNA to the blood circulation. This causes metastasis of tumors in different organs, the SSI TCM weakening the influence of autologous vaccines, prepared from the lysine of tumor cells, taken during the operation, because these vaccines do not take the tumor cell mutations, caused by the conventional triad of treatment, into account.

U.S. Pat. No. 6,702,743 titled “Ultrasound apparatus and method for tissue resonance analysis”, (2004), whose inventor is the inventor of the present patent application, teaches a Tissue Resonance Analysis (TRA). This patent discloses that when a tissue of interest is stimulated by an ultrasound pulse, the nature of the reflected ultrasound signal will depend upon the resonant state of the tissue. Therefore, by properly processing and interpreting the reflected signal, it is possible to derive information relating to the physiological state of the tissue of interest.

The TRA provides information about the physical properties of body tissue and fluids. This TRA technology is capable of monitoring the functional status of tissues anywhere in the body. Its ability to also monitor intracranial tissues and fluids constitutes a key advantage over other ultrasonic technologies whose signals cannot readily penetrate across the skull. Furthermore, the stimulation parameters, beam focusing, and sensor gates can all be modified to generate important diagnostic information about the physiological status of virtually any fluid space, tissue, or organ of interest.

In spite of all of the known and existent solutions, there is still a need for alternative therapies to overcome or mitigate at least some of the deficiencies of the prior art.

### SUMMARY OF THE INVENTION

The background art does not teach or suggest an autologous vaccine therapy, namely a vaccine derived from the person's own tumor cells that is customized for the same person, which doesn't lose effectivity as a result of mutations that occur in cancer cells over time.

Contrary to the standard practice in most allogenic cancer vaccines that also contain adjuvants, according to the present invention, a Tissue Resonance Suppression Therapy (TRST) is used in order to strengthen the immune system, and treatment with Autologous Polyvalent Biological Vaccine (APBV) is performed subsequently.

Production and administering the APBV to the patient is performed several times, at intervals, thus renewing the effectivity of the vaccine, even in the case of mutation of cancer cells.

According to the teaching of the present invention there is provided a system for the production of an autologous polyvalent biological vaccine for treating cancer, the system includes: (a) a reservoir; (b) a vaccine syringe connected to the reservoir by a vaccine syringe tubule having a vaccine syringe tap; (c) a polyprotein syringe connected to the reservoir by a polyprotein syringe tubule having a polyprotein syringe tap; (d) a mixture syringe connected to the polyprotein syringe by a mixture syringe tubule having a mixture syringe tap; (e) an alcohol vapors syringe connected to the reservoir by an alcohol vapors syringe tubule having an alcohol vapors syringe tap; and (f) an alcohol vapors production unit connected to the alcohol vapors syringe by an alcohol vapors production unit tubule having an alcohol vapors production unit tap.

According to another feature of an embodiment of the present invention, the polyprotein syringe has a polyprotein syringe micro-engine piston rod, and the alcohol vapors syringe has an alcohol vapors syringe micro-engine piston rod.

According to another feature of an embodiment of the present invention, the system for the production of an autologous polyvalent biological vaccine for treating cancer further includes: (g) a polyprotein syringe micro-engine wherein the polyprotein syringe micro-engine is connected to the polyprotein syringe micro-engine piston rod; and (h) an alcohol vapors injector micro-engine, wherein the alcohol vapors injector micro-engine is connected to the alcohol vapors syringe micro-engine piston rod.

According to another feature of an embodiment of the present invention, the polyprotein syringe contains polyprotein, wherein the mixture syringe contains mixture of immune system factors, wherein the alcohol vapors syringe contains alcohol vapors wherein the alcohol vapors production unit contains alcohol vapors, and wherein the vaccine syringe contains autologous polyvalent biological vaccine.

According to another feature of an embodiment of the present invention, the reservoir contains autologous polyvalent biological vaccine.

According to the teaching of the present invention there is provided a method for strengthening the immune response of a weakened immune system includes: (a) performing a first stage of the method for strengthening the immune response of a weakened immune system, the first stage includes: (i) providing a dose of tissue resonance suppression therapy to a patient, wherein the tissue resonance suppression therapy includes injection of mixed gas to the patient, wherein the mixed gas includes at least 40 percent of filtrated air, which includes, among others both nitrous oxide and oxygen, at least 20 percent of medical ozone at least 20 percent of medical helium, and at least 3 percent of alcohol vapors; (b) after providing at least one dose of tissue resonance suppression therapy to the patient, performing a second stage of the method for strengthening the immune response of a weakened immune system the first stage includes: (i) taking venous blood and heparin from the patient; (ii) performing sedimentation in refrigeration, in a temperature such as 4 degree Celsius and receiving a sedimented sample of erythrocytes and leucocytes; (iii) subjecting the sedimented sample to a predetermined centrifugal force; (iv) performing cell free DNA separation; (v) performing cell free DNA Terahertz spectroscopy; (vii) performing cancerous cell free DNA cloning; (viii) performing oxygenation with ozonated water at a concentration such as 5 micrograms per liter; (ix) injection of cancerous cell free DNA antibodies and mixture of reprogrammed cancer stem cells, tissue necrosis factors inhibitors, programed death 1 inhibitors, cytokine inhibitors, and anti-inflammatory protein inhibitors; and (x) Tumor cells autophagia inhibitors, inhibitors of unprogrammable tumor cell death products.

According to another feature of an embodiment of the present invention, the method includes a repeating of the second stage of the method for strengthening the immune response of a weakened immune system, several times, wherein in each of the repetition second stage, the vaccine in use is a younger generation vaccine in relation to the previous generation vaccine that was injected.

According to another feature of an embodiment of the present invention, prior to performing the method for strengthening the immune response of a weakened immune system, an evaluation is performed of the patient's immune system response to tissue resonance suppression therapy.

According to the teaching of the present invention there is provided a method for strengthening the immune response of a weakened immune system comprising: (a) performing a first stage of the method for strengthening the immune response of a weakened immune system, wherein the first stage includes: (i) providing a dose of tissue resonance suppression therapy to a patient, wherein the tissue resonance suppression therapy includes injection of mixed gas to the patient, wherein the mixed gas includes at least 40 percent of filtrated air 22, which includes, among others both nitrous oxide and oxygen, at least 20 percent of medical ozone at least 20 percent of medical helium, and at least 3 percent of alcohol vapors; (b) after providing at least one dose of tissue resonance suppression therapy to the patient, performing a second stage of the method for strengthening the immune response of a weakened immune system wherein the first stage includes: (i) injection of a first generation autologous polyvalent biological vaccine for personalized and precision medicinal treatment of cancerous patients. According to another feature of an embodiment of the present invention, the method includes a repeating of the second stage of the method for strengthening the immune response of a weakened immune system several times, wherein in each of the repetition of the second stage the vaccine in use is a younger generation vaccine in relation to the previous generation vaccine that was injected.

According to the teaching of the present invention there is provided a method of immunotherapy includes the stages of: (a) providing at least one dose of tissue resonance suppression therapy to a patient, in order to strengthen the immune system; and (b) injecting an autologous polyvalent biological vaccine to the patient, wherein the autologous polyvalent biological vaccine was produced from the patient blood.

According to another feature of an embodiment of the present invention, the method further includes the stage of: (c) after an interval of time, producing a new generation of autologous polyvalent biological vaccine from the patient blood and injecting the new generation autologous polyvalent biological vaccine to the patient.

According to the teaching of the present invention there is provided a system for performing tissue resonance suppression therapy includes: (a) a mixed gas syringe; (b) an air and alcohol filter unit connected to the mixed gas syringe by an air and alcohol filter unit tubule having an air and alcohol filter unit tubule tap; (c) an alcohol syringe connected to the air and alcohol filter unit by an alcohol syringe tubule; and (e) a needle having a needle tip connected to the mixed gas syringe by a mixed gas tubule.

According to another feature of an embodiment of the present invention, the system for performing tissue resonance suppression therapy further includes: (f) a selector connecting to the mixed gas syringe by a pipe; (g) an oxygen container connecting to the selector by a pipe having a manometer, wherein the oxygen container contains oxygen; (h) a nitrous oxide container connecting to the selector by a pipe having a manometer, wherein the nitrous oxide container contains nitrous oxide; and (i) a helium container connecting to the selector by a pipe having a manometer, wherein the helium container contains medical helium (He).

According to another feature of an embodiment of the present invention, the mixed gas syringe contains mixed gas wherein the alcohol syringe contains alcohol, wherein the air and alcohol filter unit contains air and alcohol filter, and wherein the air and alcohol filter unit is adapted to pump air into it.

According to the teaching of the present invention there is provided a process for producing does of autologous polyvalent biological vaccine for treating cancer from venous blood of an individual cancer patient, comprising the stages of: (a) removing protein surfaces wherein the proteins are taken from an individual cancer patient at a certain stage of the patient's disease, and tailoring the proteins with new surfaces; and (b) after an interval of time, producing a new dose of autologous polyvalent biological vaccine from the patient's venous blood that was taken an interval of time after when the previous venous blood was taken.

According to another feature of an embodiment of the present invention, the product is adapted for treating the specific cancer patient at the specific stage of his disease.

According to another feature of an embodiment of the present invention, the product is customized for the individual cancer patient wherein the product does not lose effectivity as a result of mutations that occur in cancer cells of the individual cancer patient over time.

### BRIEF DESCRIPTION OF THE DRAWINGS

The invention is herein described, by way of example only, with reference to the accompanying drawings, wherein:

FIG. 1 is a schematic illustration of a system for performing TRST, in accordance with an embodiment of the present invention.

FIG. 2 is a schematic illustration of a system for performing TRST, wherein its needle is inserted inside the abdomens of a patient, in accordance with an embodiment of the present invention.

FIG. 3 is a schematic illustration of a syringe, for injection of fluid into an intervertebral disc as a stage of performing TRST in a patient, in accordance with an embodiment of the present invention.

FIG. 4 is a schematic illustration of a system for production of an APBV, in accordance with an embodiment of the present invention.

FIG. 5 is a schematic illustration of some sub-stages of a first stage of producing autologous polyvalent biological vaccine in accordance with an embodiment of the present invention.

In order to leave no room for doubt, the elements are shown in the illustrations of the present patent application in a manner that enables understanding them clearly, and the scales, size relations, and shapes are not in any way limiting their embodiment.

### DETAILED DESCRIPTION OF EMBODIMENTS OF THE INVENTION

To remove any doubt, note that the manner in which the elements of the present invention are described in the illustrations can be highly detailed, however this is not in any way limiting the present invention, however is for the purpose of clarification and furthering understanding. The present invention can be implemented in embodiments that differ from the specification given with regard to the illustration.

The present invention is of a tissue resonance suppression therapy and of an autologous polyvalent biological vaccine.

The principles and operation of the tissue resonance suppression therapy and of the autologous polyvalent biological vaccine according to the present invention may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings.

Unless otherwise defined, all technical and scientific terms used herein have the same meaning as commonly understood by one of ordinary skill in the art to which this invention belongs. The numerical values, materials, dimensions, methods, and examples provided herein are illustrative only and are not intended to be limiting.

The following is a list of acronyms used in the present patent application:

AB—Antibodies

ACVT—Anti-Cancer Vaccines Therapy

AI—Artificially Induced

AIP—Anti-Inflammatory Protein

APBV—Autologous Polyvalent Biological Vaccine

b-TMR—biological-Tissue Mechanical resonant

CF DNA—Cell Free DNA

CCF DNA—Cancerous Cell Free DNA

CFP—Cell Free Peptides

COA—Chronic Osteoarthritis

CRP—C-Reactive Protein

DNA—Deoxyribonucleic Acid

EOP—Existing Overexpressed Proteins

FMA—Fibromyalgia

GIS—General Immune System

GM-CSF—Granulocyte Macrophage—Colony Stimulating factor

IDH—Intervertebral Discus Herniation

IPN—Idiopathic Peripheral Neuropathy

IL—Inter Leukin

IL-2-Leukin-2

IL-4-Leukin-2

ISF—Immune System Factors

ITPP—Intra Tissue Predetermined Pressure

IUN—International Unit

LBP—Lower Back Pain

MHC—Major Histocompatibility Complex

NHSP—Non-Heat Stress Proteins

MS—Multiple Sclerosis

NK cells—Natural Killer cells

PDI—Programmed Death 1 (regarding to cell death)

PTM—Personal Treatment Methods

STF—Treatment Factors

TCUD—Tumor Cells Un programmable Death

THZ—Terahertz

TNF—Tissue Necrotic Factors

TNFB—Tissue Necrotic Factors Blockers

TRA—Tissue Resonance Analysis

TRST—Tissue Resonance Suppression Therapy

ULA—Under Local Anesthesia

VEGF—vascular endothelial growth factor

VEGH—vascular endothelial growth hormone

The following list is a legend of the numbering of the application illustrations:
1 epidermis
2 sub-dermal fat tissue
3 superficial abdominal fascia
4 muscles of anterior abdominal wall
6 peritoneum
7 abdominal cavity
mixed gas syringe
11 mixed gas
12 mixed gas tubule
13 needle
13a needle tip
14 manometer tubule
manometer
18 fluid
19 syringe
air and alcohol filter unit
21 air and alcohol filter
22 air
23 air and alcohol filter unit tubule
24 air and alcohol filter unit tubule tap
oxygen (02)
25a oxygen container
26 nitrous oxide (N₂O)
26a nitrous oxide container
27 medical helium (He)
27a helium container
28 pipe
29 selector
alcohol syringe
30a alcohol syringe handle
31 alcohol
32 alcohol syringe tubule
33 medical ozone (03)
33a ozone generator
34 ozonated water
polyprotein syringe
40a polyprotein syringe tubule
40b polyprotein syringe tap
42 polyprotein syringe micro-engine
42a polyprotein syringe micro-engine piston rod
42b polyprotein syringe piston
polyprotein
50 alcohol vapors syringe
50a alcohol vapors syringe tubule
50b alcohol vapors syringe tap
52 alcohol vapors syringe micro-engine
52a alcohol vapors syringe micro-engine piston rod
52b alcohol vapors syringe piston
55 alcohol vapors
60 mixture syringe
60a mixture syringe tubule
60b mixture syringe tap
60c mixture syringe piston
60d mixture syringe needle
65 mixture of ISF
70 alcohol vapors production unit
70a alcohol vapors production unit tubule
70b alcohol vapors production unit tap
71 alcohol moisturized air filter
80 vaccine syringe
80a vaccine syringe tubule
80b vaccine syringe tap
80c vaccine syringe piston
80d vaccine syringe needle
85 APBV
90 reservoir
91 test tube
91a plasma
91b platelets rich plasma
91c red blood cells
92 pipette
100 system for performing TRST
200 system for production of an APBV
500 abdomens
602 spinal vertebra
603 intervertebral disc

Hereinafter, embodiments of the present invention are explained in detail by referring to the drawings.

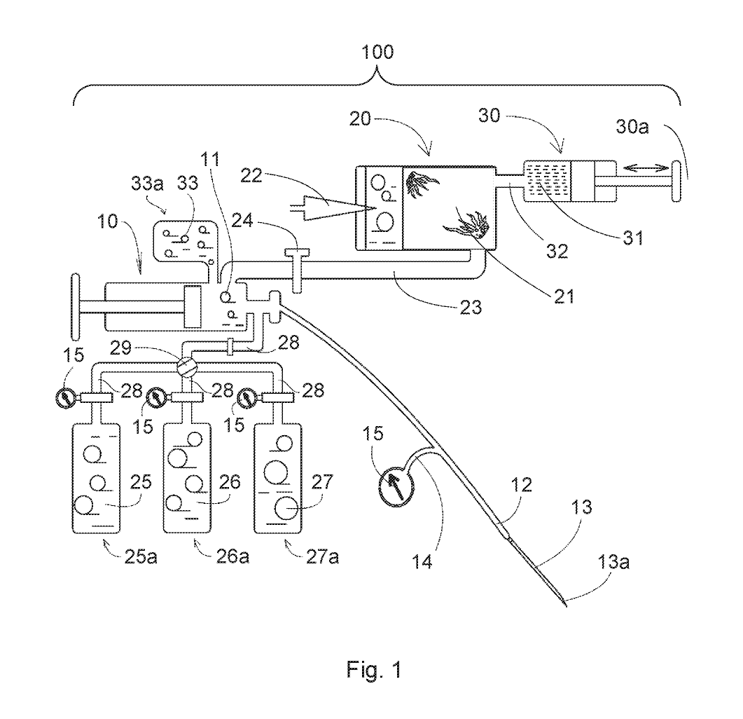

FIG. 1 is a schematic illustration of a system for performing TRST 100, in accordance with an embodiment of the present invention.

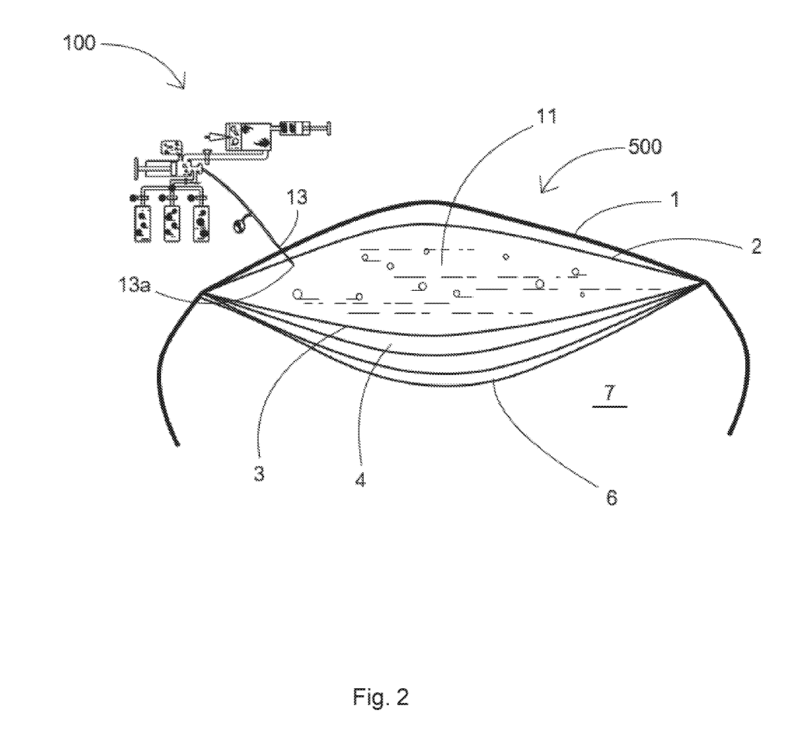

The present illustration shows one possible configuration for a system for performing TRST 100. The system for performing TRST 100 includes a mixed gas syringe 10, an air and alcohol filter unit 20, an alcohol syringe 30, a needle 13, and manometers 15. The mixed gas syringe 10 is connected to the air and alcohol filter unit 20 by an air and alcohol filter unit tubule 23 having an air and alcohol filter unit tubule tap 24. Tap 24 is closed prior to injecting the patient with mixed gas 11, as shown in FIG. 2, and prevent flow in the alcohol filter unit tubule 23.

The mixed gas 11 includes air 22, oxygen 25, medical ozone 33, nitrous oxide 26, medical helium 27, and alcohol 31 vapors.

The alcohol syringe 30 is connected to the air and alcohol filter unit 20 by an alcohol syringe tubule 32. The needle 13, is connected to the mixed gas syringe 10 by a mixed gas tubule 12 which has a needle tip 13a. A manometer 15 is connected to the mixed gas tubule 12 by a manometer tubule 14. The air and alcohol filter unit tubule 23, the alcohol syringe tubule 32, the mixed gas tubule 12, and the manometer tubule 14 are adapted to enable flow of fluids.

The manometer 15 which is connected to the mixed gas tubule 12 measure pressure at a typical range between 0.5 to 12 atmospheres.

The mixed gas syringe 10 is adapted to contain mixed gas 11. The air and alcohol filter unit 20 contains an air and alcohol filter 21, such as a cotton filter, and is adapted to receive and to contain air 22. The alcohol syringe 30 is adapted to contain alcohol 31.

Air 22 is sucked into the air and alcohol filter unit 20 when pressure is created within it that is lower than environmental atmospheric pressure, as a result of appropriate movement of the alcohol syringe handle 30a.

The system for performing TRST 100 also includes an oxygen container 25a which contains oxygen (O₂) 25 at a pressure higher than environmental pressure, a nitrous oxide container 26a which contains nitrous oxide (N₂O) 26 at a pressure higher than environmental pressure and helium container 27a which contains medical helium (Hₑ) 27 at a higher pressure than environmental pressure.

As used herein the specification and in the claims section that follows, the term “medical helium” and the like refer to helium commonly used for medical applications, at a sufficient level of sterility for such applications.

The three containers are connected by means of three pipes 28 to selector 29, with each of the three pipes 28 connected to a manometer 15, namely three manometers 15.

Selector 29 selects which gas flows and when the gas flows from every one of the three aforementioned containers into the mixed gas syringe 10 through an additional pipe 28.

The system for performing TRST 100 also includes an ozone generator 33a, which is also connected to the mixed gas syringe 10. The ozone generator 33a is a device that generates medical ozone 33.

As used herein the specification and in the claims section that follows, the term “medical ozone” and the like refer to ozone commonly used for medical applications, at a sufficient level of sterility for such applications.

FIG. 2 is a schematic illustration of a system for performing TRST 100, wherein its needle 13 is inserted inside the abdomens 500 of a patient, in accordance with an embodiment of the present invention.

At a stage of performing TRST, after inserting the needle tip 13a in between the sub-dermal fat tissue 2 and the superficial abdominal fascia 3 of the patient, mixed gas 11 is injected. Mixed gas 11, whose origin is shown in FIG. 1, does not drip through the patient epidermis 1 and the sub-dermal fat tissue 2, and does not drip into the patient abdominal cavity 7 through the patient superficial abdominal fascia 3, the patient muscles of anterior abdominal wall 4, and the peritoneum 6.

Performing TRST on a cancer patient is designated to strengthen the patient's General Immune System (GIS).

The TRST according to the present invention makes use of TRA and a novel approach to aging process and biological—Tissue Mechanical resonance (b-TMR).

Each heart beat (systole) causes distribution of blood to different body tissues. Each body tissue has its own different density of capillaries, as a result, each biological tissue has its own different mechanical resonance which result in different aging of the different tissues.

Performing the TRST includes a sub-dermal injection of the mixed gas 11. An example of an effective ratio between the components of the mixed gas 11 is: 45% filtrated air 22, which includes, among others both nitrous oxide (N₂O) 26 and oxygen (O₂) 25, 25% medical ozone (O₃) 33, 25% of medical helium (He) 27, and 5% of alcohol vapors.

All of these gases are shown in FIG. 1.

The sub-dermal injection is performed Under Local Anesthesia (ULA). The sub-dermal injection can be administered different areas of the body such as the anterior abdominal wall, the axillary areas, the posterior cervical region, and the thoracic and lumbar regions.

The injection is administered until an Intra Tissue Predetermined Pressure (ITPP) of the gas mixture 11 is achieved, the typical value of such predetermined pressure is at the range in 8 atmospheres to 12 atmospheres.

The sub-dermal injection of the mixed gas 11 causes effacement of blood circulation of lower layer and upper layer soft tissues, such us epidermis, sub-dermal fat, connective tissue, fascia, muscles, bones, vertebras, nerve fibers, and axons of peripheral nerves.

The zero-blood circulation causes full suppression of the b-TMR, and the human body of the patient recognizing a state of distress.

The TRST causes stimulation of expression of different Immune System factors (ISF), such us: Tissue Necrotic Factors (TNF), Cytokines, Prostaglandins, None Heat Stress Proteins (NHSP) with different weight (20-100 Kilo-Dalton), Collagenase Inhibitors, Calcitonin, and Endorphins. A Dalton is a standard atomic mass unit.

The elevation of the mixed ISF causes very important stimulation of the GIS of week cancer patients.

According to the present invention, prior to proceeding with the TRST, an evaluation is performed of the patient immune system response to TRST. If the patient's bone marrow does not respond to TRST with elevation of the white blood cells and with elevation of the CRP in the peripheral blood circulation, its means that the autologous polyvalent biological vaccine will be not effective for the patient.

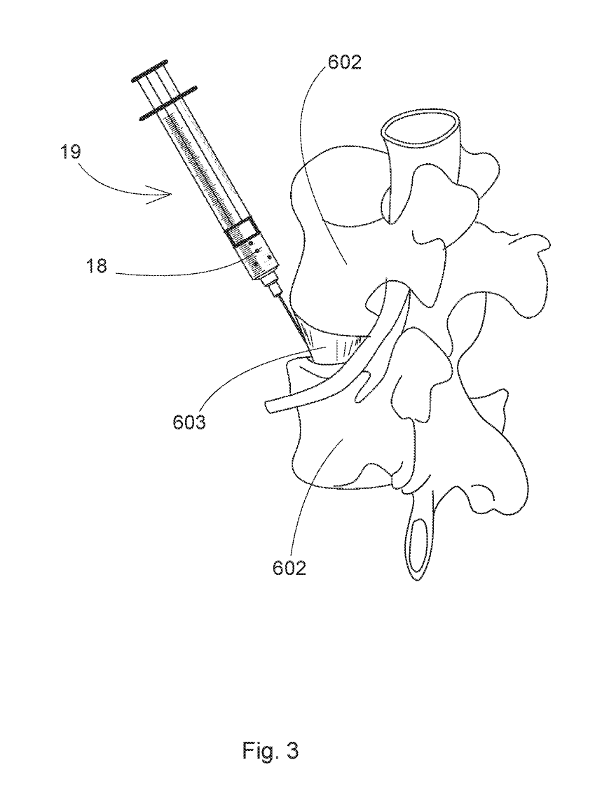

FIG. 3 is a schematic illustration of a syringe 19, for injection of fluid into an intervertebral disc 603 as a stage of performing TRST in a patient, in accordance with an embodiment of the present invention.

The intervertebral disc 603 is between two adjacent spinal vertebra 602. As noted, the injection into the intervertebral disc 603 is only one of the possible options for injection according to the present invention.

Note that TRST can serve purpose other than treatment of cancer. For example, as a drug resistant pain therapy for patients with chronic Lower Back Pain (LBP), Intervertebral Discus Herniation (IDH), Fibromyalgia (FMA), Chronic Osteoarthritis (COA), Idiopathic Peripheral Neuropathy (IPN), nervous system demyelinization diseases, Multiple Sclerosis (MS), and as immune-stimulation of human GIS.

The medical rationale for TRST, and the mechanism of performing TRST will be described in detail in the following.

Performing TRST simulates an artificial condition of transient ischemia of soft tissues at the place of injections and causes the body mistakenly recognize a state of distress at the injection site causing the release of a large quantity of Tissue Necrosis Factors (TNF) and thus causing the further cascade to release of Self Treatment Factors (STF) such us: Neuropeptides, Opiates, Macrophages, Neutrophils, Leukocytes, Lymphocytes, Interleukins, Cortisol, Noradrenalin, and Adrenalin.

The release of the STF creates stimulation and release of the body's own immune factors. This results in pain reducing effects, anti-inflammatory effects, and regeneration of different tissue, including cartilage tissue and synovial fluid.

Performing TRST also causes overexpression of the human ISF and overexpression (amplification) of the human GIS.

In patients with stage IV of cancer, very slow absorption of the air 22 and gas mixture 11, both shown in FIG. 2, causes slow release of singlet oxygen (O₁). The singlet oxygen penetrates the lymphatic system and causes much more stimulation of ISF.

After performing several cycles of treatment of TRST, such as three to five cycles, for artificial elevation of human blood plasma ISF, an amount such as 50 cubic centimeters of venous blood is taken and a mixture of ISF 65 is separated from the blood.

As used herein the specification and in the claims section that follows, the term “mixture of ISF” and the like refer to a cancerous patient's overexpressed blood plasmatic polyvalent proteins.

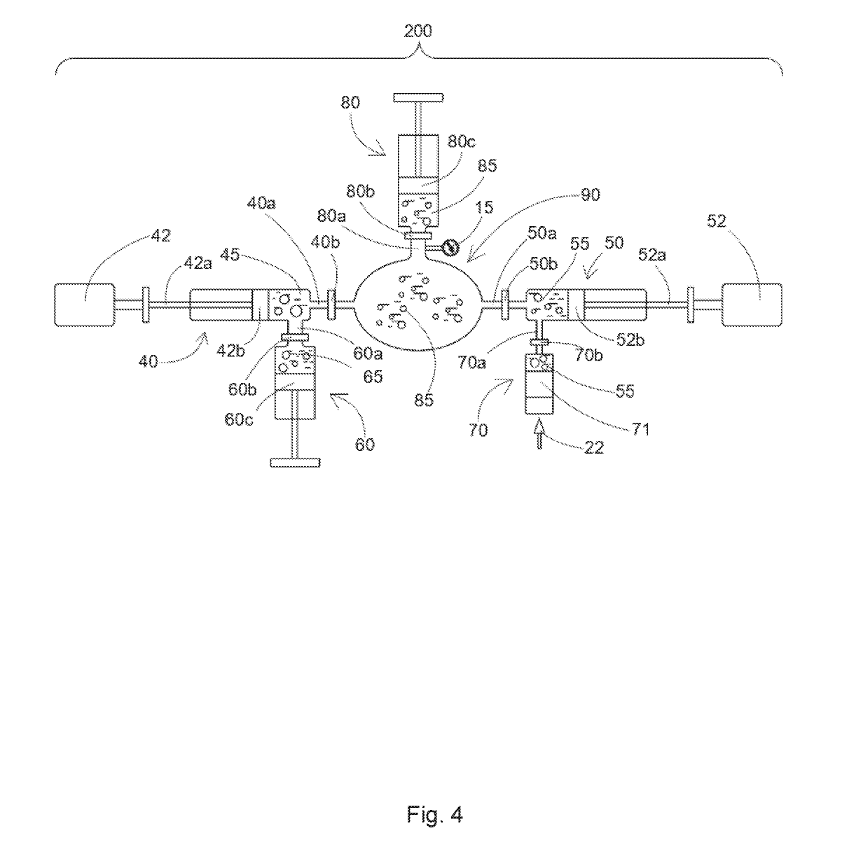

FIG. 4 is a schematic illustration of a system for production of an APBV 200, in accordance with an embodiment of the present invention.

The present illustration shows one possible configuration for a system for production of an APBV.

The system for production of an APBV 200 includes a polyprotein syringe micro-engine 42, a polyprotein syringe 40, a mixture injector 60, an alcohol vapors production unit 70, an alcohol vapors syringe 50, an alcohol vapors syringe micro-engine 52, a reservoir 90, and a vaccine syringe 80.

The polyprotein syringe micro-engine 42 is connected to a polyprotein syringe micro-engine piston rod 42a of the polyprotein mixture syringe 40, and the alcohol vapors injector micro-engine 52 is connected to an alcohol vapors syringe micro-engine piston rod 52a of the alcohol vapors syringe 50.

Both engines, the polyprotein syringe micro-engine 42 and the alcohol vapors injector micro-engine 52 work cyclically and in synchronization with each other, so that when one is applying pressure, the other one relieves pressure. Namely, when the polyprotein syringe piston 42b, which is connected to the polyprotein syringe micro-engine piston rod 42a, moves in one direction, right for example, then the alcohol vapors syringe piston 52b, which are connected to the alcohol vapors syringe micro-engine piston rod 52a, moves in the same direction, right for example, at the same time.

Thus, forces are applied alternately resulting in the mixing of the fluids within the reservoir 90.

The mixture syringe 60 is connected to the polyprotein syringe 40 by a mixture syringe tubule 60a having a mixture syringe tap 60b. The mixture syringe 60 contains a mixture syringe piston 60c.

The alcohol vapors production unit 70 is connected to the alcohol vapors syringe 50 by an alcohol vapors production unit tubule 70a having an alcohol vapors production unit tap 70b. The alcohol vapors production unit 70 contains an alcohol moisturized air filter 71 and is adapted to receive and to contains air 22.

The reservoir 90 is connected to the polyprotein syringe 40 by a polyprotein syringe tubule 40a having a polyprotein syringe tap 40b, to the alcohol vapors syringe 50 by an alcohol vapors syringe tubule 50a having an alcohol vapors syringe tap 50b, and to the vaccine syringe 80 by a vaccine syringe tubule 80a having a vaccine syringe tap 80b.

The vaccine syringe 80 contains a vaccine syringe piston 80c. A manometer 15 is connected to the vaccine syringe tubule 80a.

The mixture syringe tap 60b, the polyprotein injector tap 40b, the alcohol vapors production unit tap 70b, the alcohol vapors syringe tap 50b, and the vaccine syringe tap 80b, are all adapted to enable flow of fluids and to block flow of fluids.

After the separation of the ISF from the blood, Cancerous Cell Free DNA (CCF DNA), cancerous stem cells and cancerous maturated cells, the entire polyvalent mixture of ISF 65 is reprogrammed, using the system for production of an APBV 200.

As used herein the specification and in the claims section that follows, the term “Cancerous Cell Free DNA” and the like refer to cells which have non-programmed multiplication and non-programmed death. Also, cancerous cells have a process of autophagy (i.e. self-consumption).

As a result of this process, cell membranes are ruptured and the release of cell free DNA occurs. There are two types of existing cell free DNA: a Single strand CF-DNA also known as ssCF-DNA and a Double strand CF-DNA also known as dsCF-DNA.

The APBV 85 can be a product produced by a process that is performed with the system for production of an APBV 200.

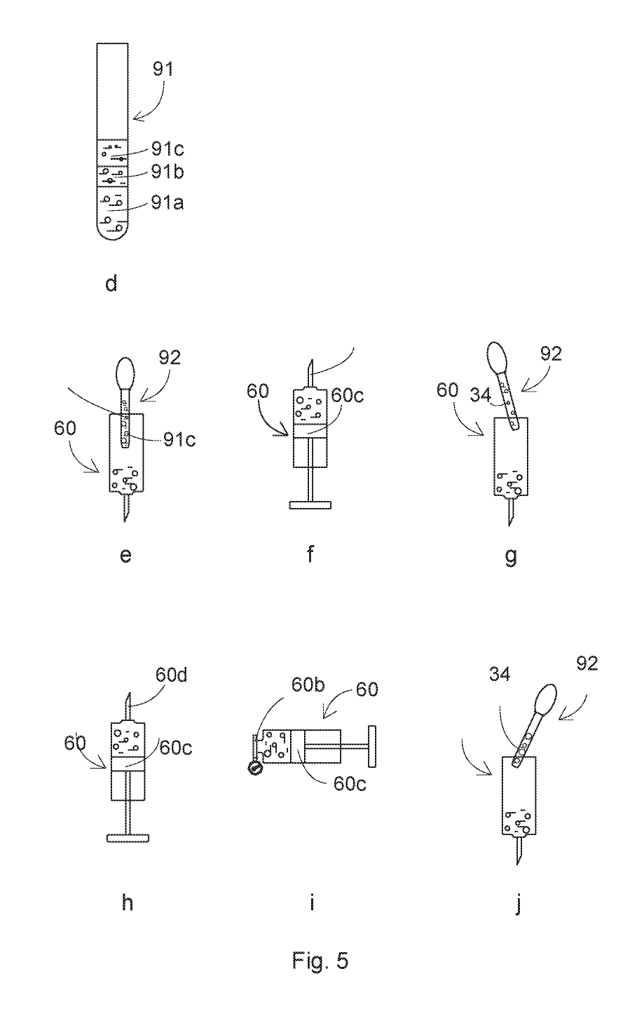

FIG. 5 is a schematic illustration of some sub-stages of a first stage of producing autologous polyvalent biological vaccine in accordance with an embodiment of the present invention.

The production process includes two stages.

In the first stage, the surfaces are removed from proteins extracted from the patient's blood, and these proteins are cased in new surfaces, so that the patient's GIS does not recognize them as the patient's own proteins, and thus the patient's GIS develops antibodies for these proteins.

In the second stage, which is primarily fortification of the new protein surface, the production of the autologous polyvalent biological vaccine is completed.

The second stage can take place in the system for production of an APBV 200 shown in FIG. 4.

The first stage includes the following sub-stages:

a. After providing three doses of TRST, taking 50 cl of venous blood, and placing it in a test tube 91;

b. Performing sedimentation of the venous blood in refrigeration for one hour, at a temperature such as 4 degrees Celsius. This process, which is performed in a containing vessel, generates a sedimentation of erythrocytes and leucocytes;

c. Subjecting the sedimented sample to a low centrifugal force, at a rotational velocity no larger than 500 rotations per minute, for approximately three minutes, with the centrifuge arm length no longer than 15 cm, or other values resulting in a similar value of centrifugal force;

d. After application of the centrifugal force, plasma 91a is accumulated at the bottom of test tube 91, immediately followed by platelets rich plasma 91b, and with red blood cells 91c at the top;

e. Using pipette 92, the platelets rich plasma 91b and the red blood cells 91c are sucked from within the test tube 91, and transferred into the mixture syringe 60;

f. A vaccine syringe piston 60c is assembled to mixture syringe 60 and is inverted in order to expel air from the mixture syringe needle 60d;

g. The mixture syringe piston 60c is removed from the mixture syringe 60, the mixture syringe 60 is inverted once again, and five drops of ozonated water 34 (O₃+H₂O) at a concentration of 5 mg/ml are added;

h. The mixture syringe piston 60c is assembled to the mixture syringe 60, and the mixture syringe 60 is inverted once more, and shaken for the purpose of mixing the contents and expelling air through the mixture syringe needle 60d;

i. The mixture syringe needle 60d is removed from the mixture syringe 60, and a mixture syringe tap 60b is assembled instead, and then the pressure within the mixture syringe 60 is raised to a range of 8-12 atmospheres. The increased pressure results in breakage and removal of the surfaces from the proteins within the mixture syringe 60 and the exposure of the vascular endothelial growth factors (VEGF);

j. Removing the mixture syringe piston 60c and adding three drops of ozonated water 34 at a concentration of 5 mg/ml into the mixture syringe 60. The result of this is protein surfaces tailoring, in which new surfaces are created for the proteins. The breakage and removal of surfaces and the surfaces tailoring applies to all of the proteins in the mixture within the mixture syringe 60. The new surfaces will be recognized as foreign by the patient's GIS, even though these proteins are from the patient's blood, and the patient's GIS will thus develop antibodies for these proteins.

In the second stage of producing of the autologous polyvalent biological vaccine 85, the newly formed surfaces are fortified. In this stage, the mixture received at the end of the first stage within the mixture syringe 60 is mixed with alcohol vapors 55. After approximately five minutes of mixing, the APBV 85 is received.

As noted, this stage can be performed by using the system for production of an APBV 200 shown in FIG. 4.

For this purpose, at the beginning of this stage, the mixture syringe 60 is assembled to its place in the system, and upon completion of mixing, the APBV 85 is sucked from the reservoir 90 in which mixing took place, by means of the vaccine syringe 80. After completion of the second stage, the patient can be injected with the APBV 85.

In the era of personalized and precision medicine, in order to perform more effective and long lasting Personal (individual) Treatment Methods (PTM) there is a need to open new avenues. According to the present invention, methods of treatment for each patient are tailored on the basis of personalized genetics; at a level of double and single strand cancerous cell free DNA, cancerous cell free DNA breakdown products (e.g. poly-peptides) as well as cellular and molecular approach.

The vaccine according to the present invention is a biological vaccine obtained from usually Existing Over-expressed Proteins (EOP) in the serum of cancer patients and from Artificially Induced (AI) over-expressed Non-Heat Stress Proteins (NHSP).

For production of personalized autologous biological polyvalent vaccines for different medical aims and applications, e.g., immunomodulation of human body for immunity stimulation, or immune rehabilitation of cancerous patients after chemotherapy and radiation therapy, for treating and prolonging survival of stage IV cancers including multiple metastasis on the different organs, including brain, spinal cord, lungs, and bones.

The APBV 85 according to the present invention includes: cytokines, IL-2, IL-4, dendritic cells, non-heat stress proteins having a molecular weight of at least 20 kilo Dalton and at most 100 kilo Dalton, tumor stem cells, endorphins, anti-inflammatory proteins, programmed death cells, non-programmed autophagic death tumor cells, double and single strand cell free DNA, cell free peptides. Tissue Necrotic Factors (TNF), Tissue Necrotic Factors Blockers (TNFB), and additional components.

The APBV 85 features a relative part of each of the components in a volume unit determined during production and is dependent on the identity of the patient and the progress of the patient's disease. Namely, every patient has an individually suitable vaccine.

After production of the APBV 85, it can be integrated into the medical protocol for treatment of cancer.

According to the present invention a method of immunotherapy is provided, the method including the stages of:

a. Providing three doses of Tissue Resonance Suppression Therapy (TRST) to a patient, in order to strengthen the immune system;

b. Injecting an Autologous Polyvalent Biological Vaccine (APBV) to the patient.

The production and administering of the APBV to the patient is performed several times, at intervals, thus renewing the effectivity of the vaccine, even in the case of mutation of cancer cells.

It is important to note that the values noted here at the stage of the method of immunotherapy are examples of effective values, however can be reasonably altered to still obtain good results.

In conclusion, the therapeutic procedure according to the present invention, is performed in two main stages. In the first stage, the cancer patient's immune system is strengthened by means of Tissue Resonance Suppression Therapy (TRST), while in the second stage, the Autologous Polyvalent Biological Vaccine (APBV) is produced and administered to the patient.

Seeing as the cancer cells mutate over time and in order to prevent a resulting decline in the efficacy of the vaccine, according to the present invention, blood is taken from the patient at intervals over an extended period of time. In the production of a typical vaccine, blood is taken approximately ten times in order to prepare a single dose of vaccine every ten days. After administering approximately fifteen doses of vaccine, the patient's immune system remembers all of the cell mutations that occurred over this period of time, and automatically creates antibodies for any other random future mutations.

Namely, different versions of vaccines are prepared, each of which will be referred to as “nth generation APBV”, with n being a positive whole number. Thus, there is a first generation APBV, a second generation APBV, etc.

Likewise, nth generation treatments are subsequently administered, resulting in a vaccine that is effective over time.

The aforementioned numbers in this description are in no way limiting the present invention.

While the invention has been described with respect to a limited number of embodiments, it will be appreciated that many variations, modifications and other applications of the invention may be made.

What is claimed is:

1. A system for the production of an autologous polyvalent biological vaccine for treating cancer, the system comprising:
(a) a reservoir;
(b) a vaccine syringe connected to said reservoir by a vaccine syringe tubule having a vaccine syringe tap;
(c) a polyprotein syringe connected to said reservoir by a polyprotein syringe tubule having a polyprotein syringe tap;
(d) a mixture syringe connected to said polyprotein syringe by a mixture syringe tubule having a mixture syringe tap;
(e) an alcohol vapors syringe connected to said reservoir by an alcohol vapors syringe tubule having an alcohol vapors syringe tap; and
(f) an alcohol vapors production unit connected to said alcohol vapors syringe by an alcohol vapors production unit tubule having an alcohol vapors production unit tap.

2. The system for the production of an autologous polyvalent biological vaccine for treating cancer of claim 1, wherein said polyprotein syringe has a polyprotein syringe micro-engine piston rod, and said alcohol vapors syringe has an alcohol vapors syringe micro-engine piston rod.

3. The system for the production of an autologous polyvalent biological vaccine for treating cancer of claim 2, further comprising:
(g) a polyprotein syringe micro-engine wherein said polyprotein syringe micro-engine is connected to said polyprotein syringe micro-engine piston rod; and
(h) an alcohol vapors injector micro-engine, wherein said alcohol vapors injector micro-engine is connected to said alcohol vapors syringe micro-engine piston rod.

4. The system for the production of an autologous polyvalent biological vaccine for treating cancer of claim 3, wherein said polyprotein syringe contains polyprotein, wherein said mixture syringe contains mixture of immune system factors, wherein said alcohol vapors syringe contains alcohol vapors, wherein said alcohol vapors production unit contains alcohol vapors, and wherein said vaccine syringe contains autologous polyvalent biological vaccine.

5. The system for the production of an autologous polyvalent biological vaccine for treating cancer of claim 3, wherein said reservoir contains autologous polyvalent biological vaccine.

6. A method for strengthening the immune response of a weakened immune system comprising:
(a) performing a first stage of said method for strengthening the immune response of a weakened immune system, said first stage includes:
(i) providing a dose of tissue resonance suppression therapy to a patient, wherein said tissue resonance suppression therapy includes injection of mixed gas to said patient, wherein said mixed gas includes at least 40 percent of filtrated air, which includes, among others both nitrous and oxygen, at least 20 percent of medical ozone at least 20 percent of medical helium, and at least 3 percent of alcohol vapors;
(b) after providing at least one dose of tissue resonance suppression therapy to said patient, performing a second stage of said method for strengthening the immune response of a weakened immune system said second stage includes:
(i) taking venous blood and heparin from said patient;
(ii) performing sedimentation in refrigeration, in a temperature such as 4 degree Celsius and receiving a sedimented sample of erythrocytes and leucocytes;
(iii) subjecting said sedimented sample to a predetermined centrifugal force;
(iv) performing cell free DNA separation;
(v) performing cell free DNA Terahertz spectroscopy;
(vii) performing cancerous cell free DNA cloning;
(viii) performing oxygenation with ozonated water at a concentration such as 5 micrograms per liter;
(ix) injecting of cancerous cell free DNA antibodies and mixture of reprogrammed cancer stem cells, tissue necrosis factors inhibitors, programed death 1 inhibitors, cytokine inhibitors, and anti-inflammatory protein inhibitors; and
(x) performing tumor cells autophagia inhibitors, inhibitors of unprogrammable tumor cell death products.

7. Repeating said second stage of said method for strengthening the immune response of a weakened immune system, of claim 6 several times, wherein in each of said repetition second stage, the vaccine in use is a younger generation vaccine in relation to the previous generation vaccine that was injected.

8. Before performing said first stage of said method of claim 6, performing an evaluation of the patient immune system response to tissue resonance suppression therapy.

9. A method for strengthening the immune response of a weakened immune system comprising:
(a) performing a first stage of said method for strengthening the immune response of a weakened immune system, wherein said first stage includes:
(i) providing a dose of tissue resonance suppression therapy to a patient, wherein the tissue resonance suppression therapy includes injection of mixed gas to said patient, wherein said mixed gas includes at least 40 percent of filtrated air 22, which includes, among others both nitrous oxide and oxygen, at least 20 percent of medical ozone at least 20 percent of medical helium, and at least 3 percent of alcohol vapors;
(b) after providing at least one dose of tissue resonance suppression therapy to said patient, performing a second stage of said method for strengthening the immune response of a weakened immune system wherein said first stage includes:
(i) injection of a first generation autologous polyvalent biological vaccine for personalized and precision medicinal treatment of cancerous patients.

10. Repeating said second stage of said method for strengthening the immune response of a weakened immune system of claim 9 several times, wherein in each said repetition of said second stage the vaccine in use is a younger generation vaccine in relation to the previous generation vaccine that was injected.

11. Before performing said first stage of said method of claim 9, performing an evaluation of the patient immune system response to tissue resonance suppression therapy.

12. A method of immunotherapy comprising the stages of:
(a) providing at least one dose of tissue resonance suppression therapy to a patient, in order to strengthen the immune system; and
(b) injecting an autologous polyvalent biological vaccine to said patient, wherein said autologous polyvalent biological vaccine was produced from said patient blood.

13. The method of claim 13 further comprising the stage of:
(c) after an interval of time, producing a new generation of autologous polyvalent biological vaccine from said patient blood and injecting said new generation autologous polyvalent biological vaccine to said patient.

14. A system for performing tissue resonance suppression therapy comprising:
(a) a mixed gas syringe;
(b) an air and alcohol filter unit connected to said mixed gas syringe by an air and alcohol filter unit tubule having an air and alcohol filter unit tubule tap;
(c) an alcohol syringe connected to said air and alcohol filter unit by an alcohol syringe tubule; and
(e) a needle having a needle tip connected to said mixed gas syringe by a mixed gas tubule.

15. The system for performing tissue resonance suppression therapy of claim 14 further comprising:
(f) a selector connecting to said mixed gas syringe by a pipe;
(g) an oxygen container connecting to said selector by a pipe having a manometer, wherein said oxygen container contains oxygen;
(h) a nitrous oxide container connecting to said selector by a pipe having a manometer, wherein said nitrous oxide container contains nitrous oxide; and
(i) a helium container connecting to said selector by a pipe having a manometer, wherein said helium container contains medical helium (He).

16. The system for performing tissue resonance suppression therapy of claim 14, wherein said mixed gas syringe contains mixed gas wherein said alcohol syringe contains alcohol, wherein said air and alcohol filter unit contains air and alcohol filter, and wherein said air and alcohol filter unit is adapted to pump air into it.

17. A process for producing does of autologous polyvalent biological vaccine for treating cancer from venous blood of an individual cancer patient, comprising the stages of:
(a) removing protein surfaces wherein said proteins are taken from an individual cancer patient at a certain stage of said patient's disease, and tailoring said proteins with new surfaces; and
(b) after an interval of time, producing a new dose of autologous polyvalent biological vaccine from said patient's venous blood that was taken at interval of time after the time that the previous venous blood was taken.

18. The product of the process of claim 17, wherein said product is adapted for treating said specific cancer patient at said specific stage of his disease.

19. The product of the process of claim 17, wherein said product is customized for said individual cancer patient wherein said product does not lose effectivity as a result of mutations that occur in cancer cells of said individual cancer patient over time.
